(19) United States
(12) Patent Application Publication
(10) Pub. No.: US 2011/0106159 A1
(43) Pub. Date: May 5, 2011

(54) SPINAL FIXATION PLATE ASSEMBLY

(75) Inventor: Benjamin M. Nazeck, San Clemente, CA (US)

(73) Assignee: SEASPINE, INC., Vista, CA (US)

(21) Appl. No.: 12/995,995

(22) PCT Filed: Jun. 5, 2008
(86) PCT No.: PCT/US2008/007074

§ 371 Date: Dec. 2, 2010

(51) Int. Cl. A61B 17/70

(52) U.S. Cl. 606/246

(57) ABSTRACT

A spinal fixation plate has inner and outer surfaces through which a pair of adjacent screw holes extend along generally parallel axii. The screw holes accommodate the heads of conventional bone screws while allowing the threaded shafts of the screws to pass therethrough. A recess extends into each screw hole for receiving a V-shaped latch having a pair of wings joined to a head via elastically deformable arms. The latch is moveable between a deployed position in which the wings extend into the screw holes to prevent a screw seated therein from backing out and a retracted position in which the wings arc retracted from the screw holes enabling the screws to be backed out of the plate.

### TECHNICAL FIELD

The present invention relates to a plate system or assembly, in conjunction with pedicle or bone screws, for aligning and maintaining adjacent cervical vertebrae in a desired spinal relationship during spinal fusion of the vertebrae.

### BACKGROUND OF THE INVENTION

Current practice in the art of cervical spinal fusion is to secure a cervical plate along two or more vertebral bodies through the use of bone screws which extend through screw holes formed in the plate. The fixation plate, as installed, serves to immobilize the vertebral bodies. This immobilization when employed with bone graft promotes the occurrence of fusion between the adjacent vertebral bodies. The goal of this procedure is to restore an appropriate disc height between the bodies and reduce the patient's pain.

Several challenges exist in the use of cervical spinal fusion plates. One, the screws employed to secure the plate to the underlying bone have a tendency to accidentally back out of the plate over time due to motion of the underlying bone segments. This backing out phenomenon will result in instability of the joint and pain for the patient. In addition, such backing out, may cause the screws and/or plate to come into unwanted contact with soft tissue and the esophagus.

Two, there are occasions when the surgeon needs to be able to reset one or more of the screws into a different location in the underlying bone or replace the plate. Thus, any back out prevention arrangement between the screws and the plate must allow the screws to be purposely backed out.

Three, since cervical fixation plates are necessarily surrounded by soft tissue and located in close proximity to the esophagus it is highly desirable for the plates to have a low profile, i.e., have a minimum thickness, while meeting the strength requirements. It is also preferable to keep the plates as narrow as possible to reduce the chances that the lateral edges of the plate will rise off of the underlying vertebral body and cause pain/dysphagia where the curvature of the plate does not exactly match the patient's anatomy.

The back out problem has been addressed in a variety of ways. See Cordaro, U.S. Pat. No. 7,220,263 which discloses a screw and cervical plate design in which the entry section of the screw hole includes a partial thread followed by a larger diameter neck section so that the screw head, once advanced into the neck section, can rotate freely to allow the plate to be pulled tightly against the underlying bone during surgery. Once installed, the threaded entry section prevents the screw from being pushed out by motion of the underlying bone, but permits the screw to be rotated out of the screw hole. While this design prevents a common screw back out problem, i.e., the screw being pushed out, it does not eliminate the possibility that movement of the underlying bone will cause the screw to rotate in a counterclockwise direction and in this manner back out of the plate.

Also see Michelson, U.S. Pat. No. 6,936,050, to which discloses the use of locking screws or removable rivets which overlay the heads of installed pedicle screws. This type of locking arrangement does not readily lend itself to a low profile contoured surface suitable for deployment on the anterior aspect of the spine. In addition, the locking screws add complexity to the system and are not particularly surgeon friendly. Also, see Koo, U.S. Patent Publication No. 2005/0021032 which teaches the use of a plate screwed over the heads of the installed screws by rivets.

Binder, publication U.S. 2005/0234455, teaches the use of an interference fit between the pedicle screw heads and the plate screw holes to prevent screw back out on the premise that the normal torsional and bending motion of the body and spine will not cause the screws to overcome the friction resulting from the interference fit. As a precautionary matter, the inventor advises the use of set screws. Without the additional set screws the system does not appear to be fail safe from a back out standpoint and with the addition of the screw screws has the same shortcomings as the Michelson system. In addition, it is customary for the fixation plates to allow the bone screws to assume different angles relative to the longitudinal axis of the screw holes, i.e., swivel relative to the plate to allow the surgeon to penetrate the vertebral bodies at an optimum angle. The Binder system would not seem to accommodate such angulation.

Several prior art patents teach the use of slidable plates which are arranged to extend over the tops of the installed pedicle screws. See Richelsoph et al, U.S. Pat. No. 6,695,846; Paul, U.S. Pat. No. 7,008,426; Lin, U.S. Patent Publication No. 2005/0261689; Baynham, U.S. Publication No. 2005/0177161; Paul et al, U.S. Pat. No. 6,755,833 and Assaker et al, U.S. Pat. No. 6,652,525.

Several prior art approaches involve the use of a spring member such as an elongated retainer inserted over the installed screw heads and held in place by recess located below the anterior surface of the plate. See Ferree, U.S. Pat. No. 7,025,769. Other approaches involve the use of split elastically deformable rings carried by a recess in the screw head or in the plate surrounding the screw hole. See Fallin et al, U.S. Pat. No. 7,309,340 (“340 patent”); Mosca et al, U.S. Publication No. 2005/0192577; Lindemann et al, U.S. Publication No. 2005/0283152; Blain, U.S. Publication No. 2006/0235412; and Campbell et al, U.S. Pat. No. 6,602,255 (“'255 patent”).

The '340 patent discloses an anti-backout cervical plate/screw system in which a split ring is carried in a recess in the head of the screw and expends out to rest beneath an undercut in the plate surrounding the screw hole when installed. The plate has access channels spaced around the screw hole through which the prongs of a removal tool can be inserted to compress the split ring so that the screw can be removed. A similar anti-backout arrangement is used in a prior art plate/screw system marketed by the assignee of this application, SeaSpine, Inc., under the brand name Sonoma Plate.

Cervical plate systems with an anti-backout feature which relies on an undercut around all or most of the screw holes, as in the '340 and Sonoma Plate systems, compromise the low profile criteria by requiring a greater thickness in the region of the undercut. The split ring arrangement of the '255 patent undercuts and thus compromises the medial area between the crew holes. Such area is critical to the overall strength of the plate. In addition, the locking mechanism of the '255 patent reduces the effective area of the graft window and compromises the ability of the surgeon to visually verify correct graft placement and compression.

There is a need for an improved fixation plate assembly or system which more closely meets the challenges discussed above.

### SUMMARY OF THE INVENTION

A spinal fixation plate assembly in accordance with the present invention includes a fixation plate having an anterior surface, a posterior surface and at least one pair of spaced screw holes extending through the surfaces along substantially parallel axii bisected by a central perpendicular plane. Each screw hole has a generally cylindrical wall to allow the threaded shaft of a bone screw to pass therethrough, but not the enlarged head. The screw hole wall accommodates the head of the screw with a latch engaging surface of the screw head (which may be the top of the head) located below a lateral plane generally perpendicular to the associated longitudinal axis.

A v-shaped latch member or latch having a central head portion joined to a pair of wings through elastically deformable arms is carried by the plate, for example, in a recess or cavity. The latch is moveable between a deployed and a retracted position. In its deployed position the wings extend into the screw holes above the lateral plane to substantially prevent a screw positioned in the hole from backing out. In its retracted position the wings are retracted from the screw holes to allow the screw to be removed.

The application of a force to the latch head in a direction away from the screw holes serves to move the latch from its deployed to its retracted position.

Preferably the latch head is formed with a tool engaging surface and the plate is formed with a tool access opening through which a tool may be inserted to apply force to the tool engaging surface of the latch head. Alternatively, a tool may be inserted into the screw hole on top of the screw head to push a latch wing out of the way.

Additional features of the invention are discussed in the preferred embodiment section, the claims and shown in the drawings. The construction of the spinal fixation plate assembly of the present invention and the various features thereof may best be understood in reference to the following description taken in conjunction with the appended drawings.

### BRIEF DESCRIPTION OF THE DRAWINGS

FIG. 1 is top plan view of a spinal fixation plate assembly designed to span two vertebrae, i.e., employing 4 screw holes, in accordance with the present invention;

FIG. 2 is a perspective view of the disassembled plate assembly of FIG. 1;

FIG. 3 is a top plan view of the pate assembly of FIG. 1 with the latch cover plates removed and a latch in its deployed position in the left hand recess;

FIG. 4 shows the latch per se in a plan and side view;

FIG. 5 is a perspective view of the plate assembly with latch cover removed from the left side recess, showing the latch in its retracted position and the latch retracting tool;

FIG. 6 is a top plan view of the plate assembly with the actuating end of the latch retracting tool (shown in cross-section) inserted into the access opening;

FIG. 7 is a cross-sectional view of the plate assembly of FIG. 1 taken along lines 7-7;

FIG. 8 is an end view of the plate assembly of FIG. 1;

FIG. 9 is a side elevational and top plan view of a typical pedicle or bone screw suitable for use in installing the plate assembly;

FIGS. 10a, 10b, 10c and 10d are enlarged cross-sectional views taken along lines 10-10 with the head of the screw positioned slightly above the latch wind (10a) engaging the latch wing to retract the same (10b, 10c) and fully inserted into the hole with the latch wing overlying the latch engaging surface of the screw head (10d);

FIG. 11 is a top plan view of a spinal fixation plate having six screw holes designed to span three vertebral bodies;

FIGS. 12a and 12b are top plan views of a modified six hole spinal fixation plate, respectively, with FIG. 12a showing the latches in place;

FIG. 13 is a cross-sectional view of the upper portion of the plate taken along lines 13-13 of FIG. 12 showing the recess for receiving the latch;

FIG. 14 is a plan view of the upper portion of the plate of FIG. 12 showing, in dashed line, the undercut portions of the plate forming an overhang above the recess to secure the latch in place;

FIG. 15a is an enlarged view of the plate portion of FIG. 14 more clearly showing the undercut;

FIG. 15b is a cross-sectional view of the plate portion taken along lines 15b;

FIGS. 16a and 16b are plan views of a latch in its natural and compressed state (for insertion into the plate cavity) respectively;

FIGS. 17a and 17b are plan views of the upper end of the plate showing a latch ready for insertion into the cavity (FIG. 17a) and in place (FIG. 17b); and

FIGS. 18a and 18b are cross-sectional views of the plate of FIGS. 17a and 17b, respectively, showing the latch being installed into the plate cavity.

### DESCRIPTION OF THE PREFERRED EMBODIMENT

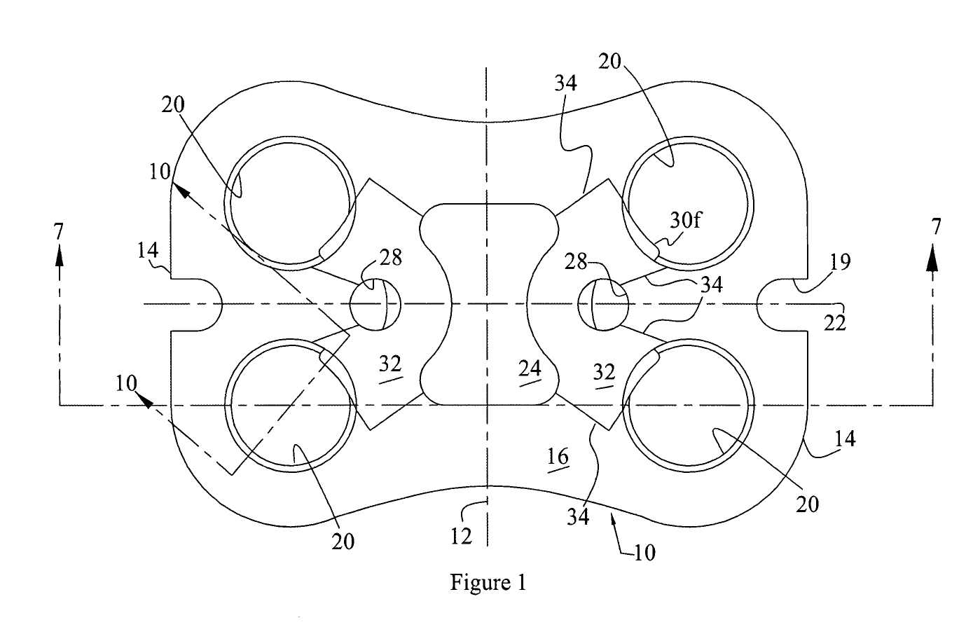

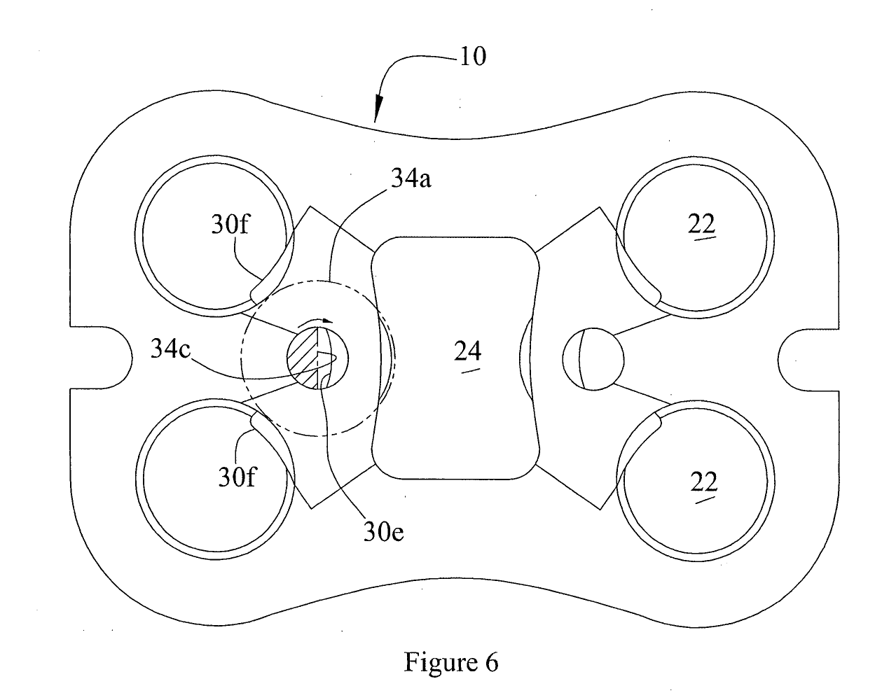

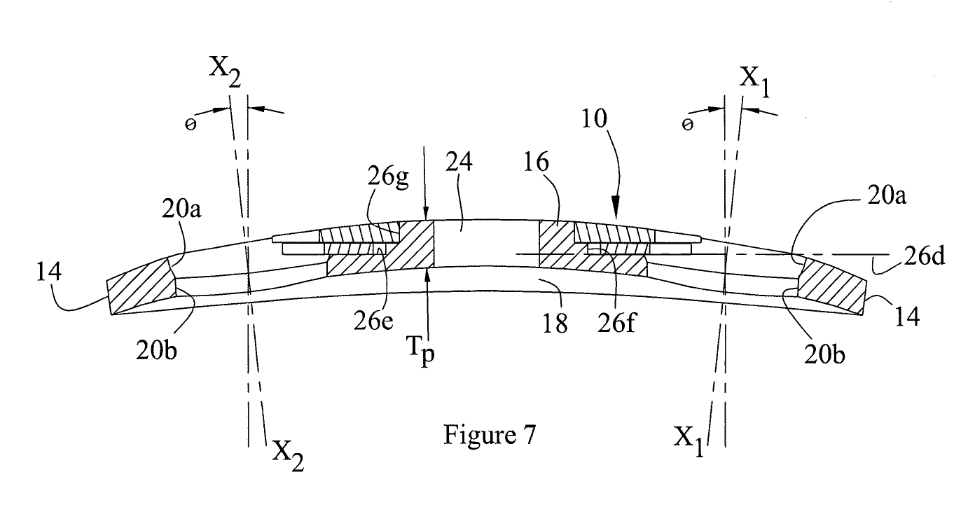

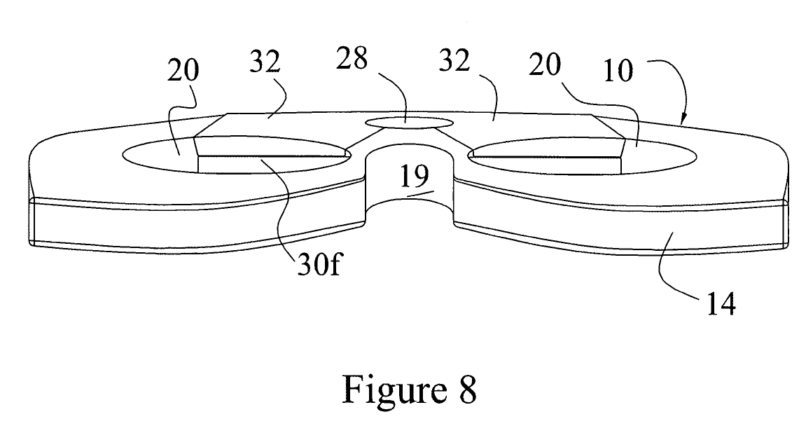

Referring now to the drawings, and particularly to FIGS. 1-7, a spinal fixation plate assembly, in accordance with the present invention, comprises a plate 10 and a latch to be described, (only a portion of the latch is shown in FIG. 1). The plate 10 is generally rectangular in shape and symmetrically formed about a midline 12 with two ends 14 having a slightly greater width than that at the midline. The plate has an anterior or inner surface 16, facing a patient's soft tissue and esophagus when installed and a posterior or outer surface 18 (FIG. 7) facing the vertebral bodies to be immobilized. A notch 19 is formed in each end of the plate. Four openings or screw holes 20 are arranged geometrically at the four corners of the rectangular plate and bisected by a vertical plane 22 as shown. The central portion of the plate defines a bone graft window 24. The two portions of the plate on each side of the midline 12 are mirror images of each other. Each of the screw holes extend through the surfaces along an imaginary longitudinal axis. The axii for two of the holes on opposite ends of the plate are designated by X1 and X2 on FIG. 7. The plate 10 forms a compound curve from end to end and side to side (i.e., convex on the anterior surface) to follow the contour of the spinal region for which the plate is designed (see FIGS. 7 and 8) so that the longitudinal axii of each pair of adjacent screw holes diverge slightly as they extend outwardly from the anterior surface. By the same token the longitudinal axii of the screw holes located on the same end of the plate diverge slightly as they extend outwardly from the anterior surface.

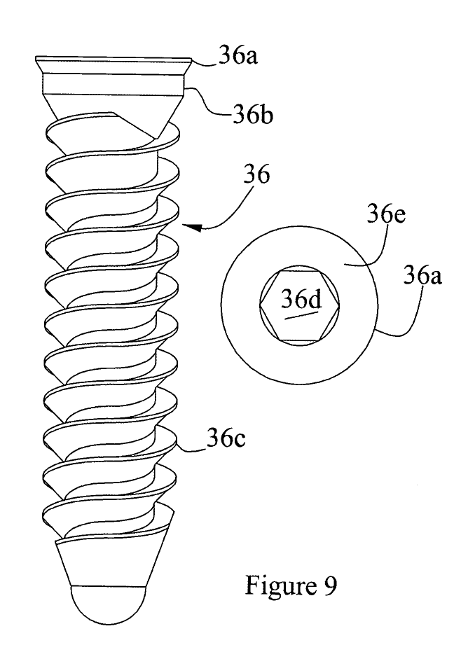

Reference is now directed to one of the screw holes 20, as shown in the cross-sectional view of FIG. 7 (since all of the screw holes are identical except for their orientation). The screw hole 20 has a generally cylindrical interior wall or surface with an upper section 20a preferably having a spherical or radiused contour for accommodating the enlarged spherical or radiused head of a conventional pedicle or bone screw therein and a lower neck section 20b, the diameter which is smaller than that of the upper section to allow the threaded shaft of the screw to pass there through, but not the head. A conventional bone screw is shown in FIG. 9 and will be described in more detail.

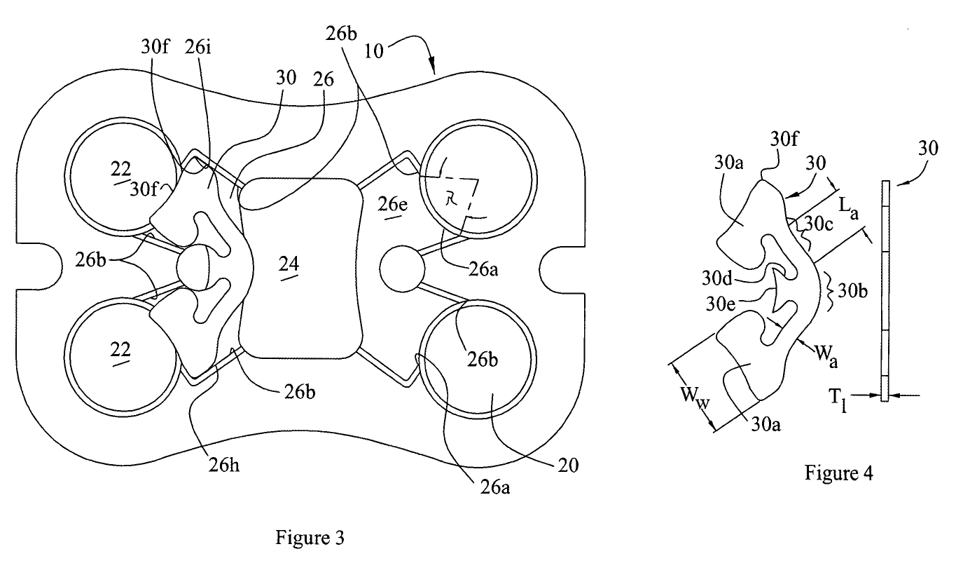

The plate is formed with a pair of planar latch supporting depressions or recesses 26 with stepped inner and outer walls 26a and 26b, respectively (FIG. 3), which extend from the bone graft window 24 to intersect the respective pair of screw holes along an arc beginning at a proximal point 26c near the central plane 22 and terminating at a distal point 26d remote from the central plane. The sector encompasses an included angle A, (FIG. 3) within the range of about 40° to 80° and preferably about 75°.

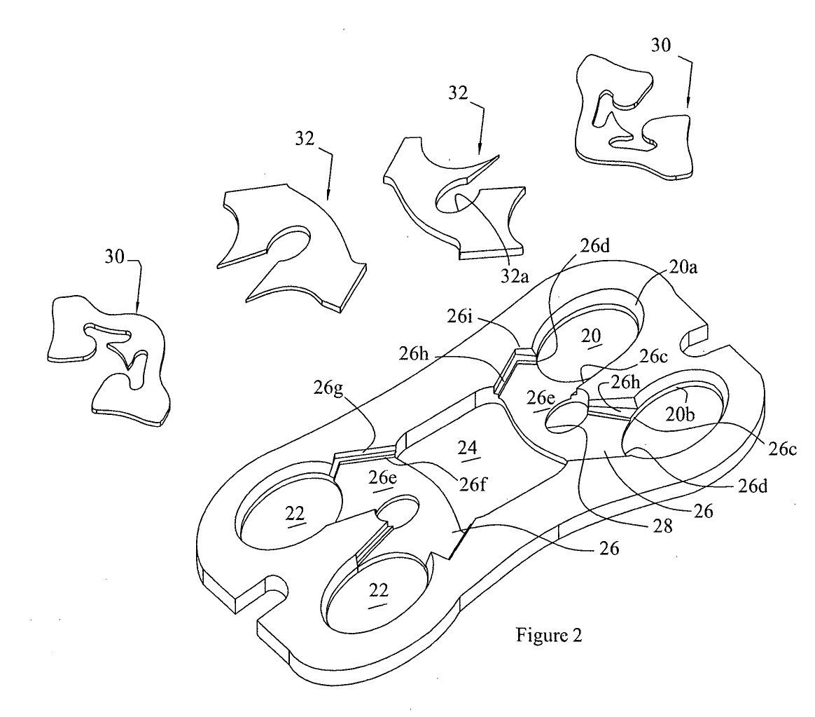

Each of the walls 26a and 26b extends upwardly from a floor 26e along a lower and upper portions 26f and 26g, respectively, to form an intermediate shelf 26h (FIGS. 2 and 7). The plate further defines a latch access opening or through hole 28. A latch 30 (FIGS. 2 and 4) is positioned on the depressed floor 26e and covered by a latch cover 32 (forming a part of the plate 10) seated on the shelf 26h and secured to the top of the plate by welding, for example, along lines 34 (FIG. 1). The latch cover 32 includes an opening 32a which is aligned with and completes the through hole 28 when the assembly is complete. The lines 34 representing the weld lines would undoubtedly disappear in a commercially finished plate assembly. The recesses 26 and the latch cover form cavity 26/32 for retaining a latch to be described.

Referring again to FIG. 4, the latch 30, is generally v-shaped with a pair of inwardly extending wings 30a, joined to a central head portion 30b through a pair of arms 30c. An inwardly projecting post 30d, forming part of the head, includes a tool engaging surface 30e. The latch is made of a high strength elastically deformable or resilient material such as a nickel titanium alloy, i.e., Nitinol. The plate is made of a high strength bio-compatible material such as titanium or stainless steel. The cross-sectional area of the arms 30c in conjunction with their lengths provide most of the elastic deformation when the latch is placed under stress as will be explained. For example, where the width W_{w} of the wing is 0.117″, the width Wₐ of the arm may be 0.030″ with a length Lₐ of 0.080″. The thickness T₁ of the latch may be 0.013″ and the overall thickness Tp (FIG. 7) of the plate may be about 1.5 mm.

Referring now to FIG. 3, a latch 30 is positioned in the recess 26 on the left hand side of the plate with the latch covers 32 removed. As depicted, the latch is in its natural state or deployed position, i.e, in an unstressed or preferably in a slightly stressed state. A large portion of the outer most end or tip 30f of each of the wings 30a protrudes into the associated screw hole as shown.

The lower outer wall 26g terminates in a corner 26i against which the inner corner 30f of the latch wing abuts in its natural state as is illustrated in FIG. 3. The corner 26i serves to limit the movement of the wing into the screw hole and in conjunction with the angled orientation of the wall 26g causes the wing to rotate as the latch is retracted.

FIG. 5 illustrates the latch being moved into its retracted position as a result of a force being applied against the tool engaging surface 30e away from the respective screw holes as shown. The force simultaneously (1) moves the latch inwardly (exposing more of the head in the graft window) (2) flexes the elastically deformable arms inwardly, and (3) rotates the heads toward each other, retracting the wings from the respective screw holes as is illustrated by the arrows.

A simple latch retracting tool 34 is shown above the plate 10 in FIG. 5. This tool includes a manually rotatable knob 34a and a shaft 34b terminating in a semi-cylindrical end 34c.

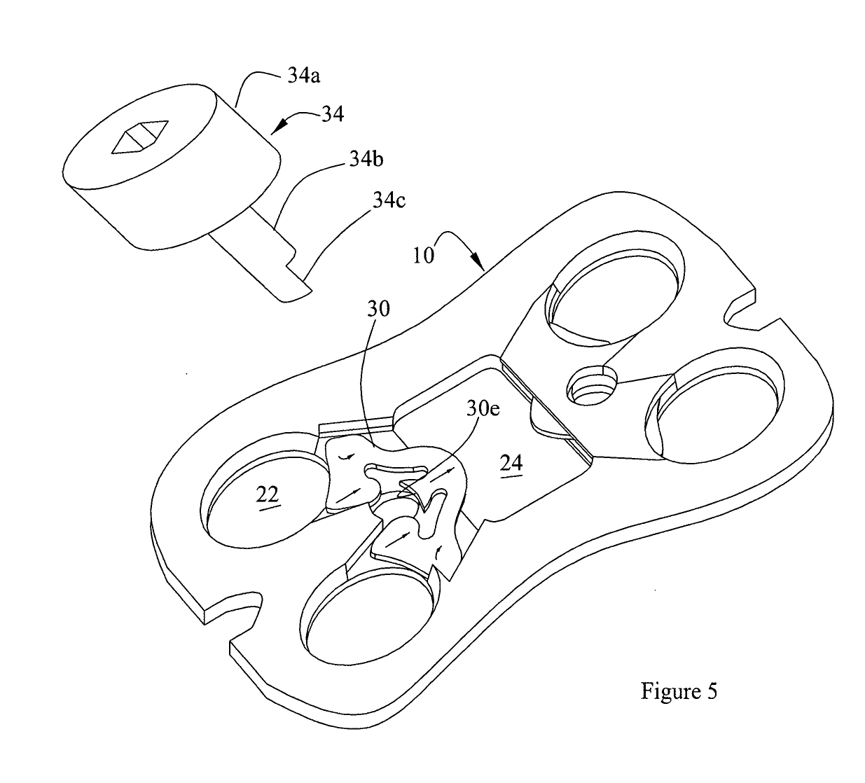

FIG. 6 illustrates a complete fixation plate assembly with the tool 34 inserted into the latch access opening. The tool's terminal end is shown in cross-section. Rotation of the tool will force the free end 34e of the post 34d out of the access opening to move the latch into its retracted position.

FIG. 9 shows a conventional bone screw 36 with an enlarged head 36a which is typically spherical or radiused followed by a generally cylidrical neck portion 36b and a threaded shaft 36c. An insertion/removal tool engaging surface 36d extends into the top of the screw head for accommodating an alien wrench. The top of the screw head surrounding the tool engaging surface forms a latch engaging surface 36e.

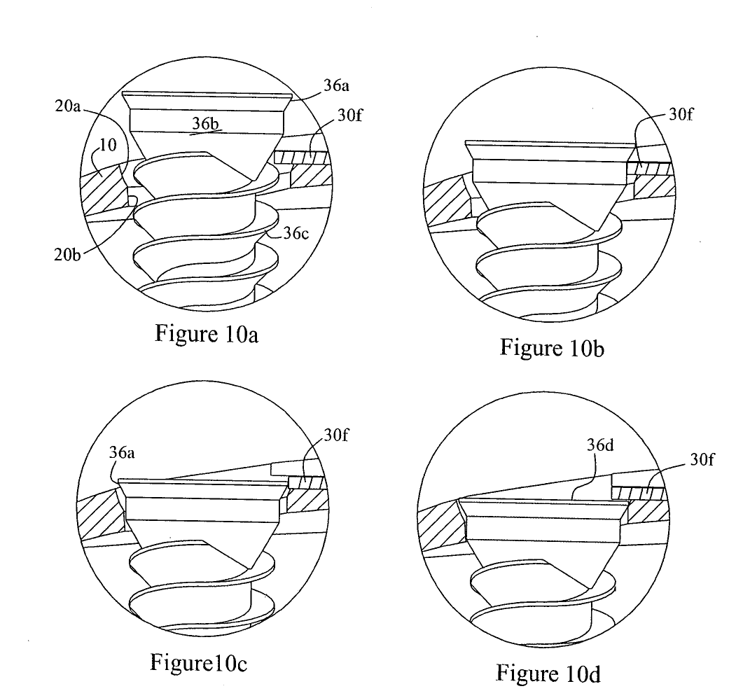

The progression of the screw head into the screw hole pushing the latch wing tip 30f into its retracted position is illustrated in FIGS. 10a-d. FIG. 10a shows a tapered portion of the shaft, leading to the neck 30b contracting the wing tip 30f. FIGS. 10b and 10c show the screw neck and head forcing the tip 30f back out of the hole. FIG. 10d shows the screw head fully seated in the screw hole with the wing tip extending over the top (latch engaging surface) of the screw. The bottom surface of the wing tip, being normal to the top surface of the screw, prevents the screw from accidentally backing out of the plate.

FIG. 11 illustrates a six screw hole spinal fixation plate 10′ with two bone graft openings. This arrangement is identical to that of FIG. 1 except for the additional screw holes and bone graft opening with like elements marked with a prime numeral. It is to be noted that sometimes in the following claims the term “plate” is used to encompass the latch.

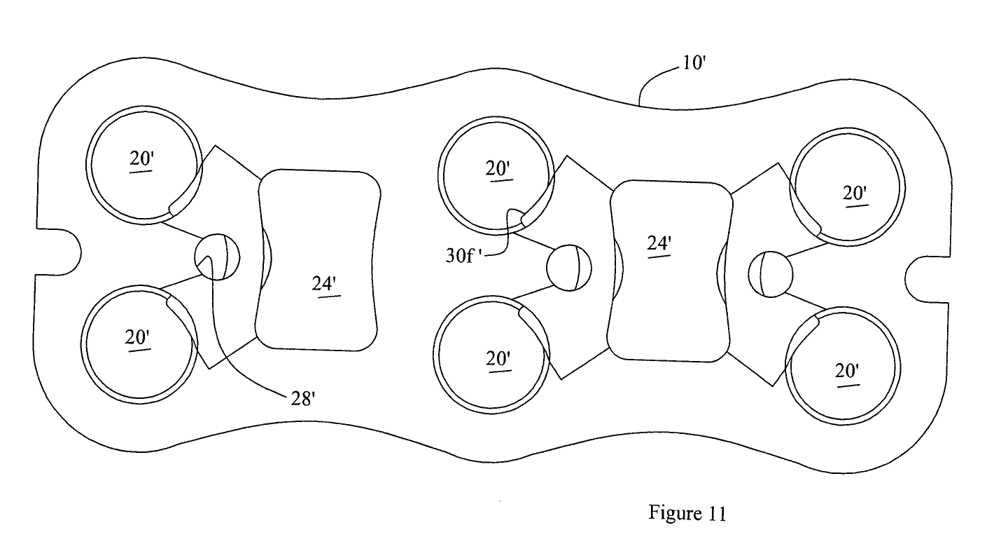

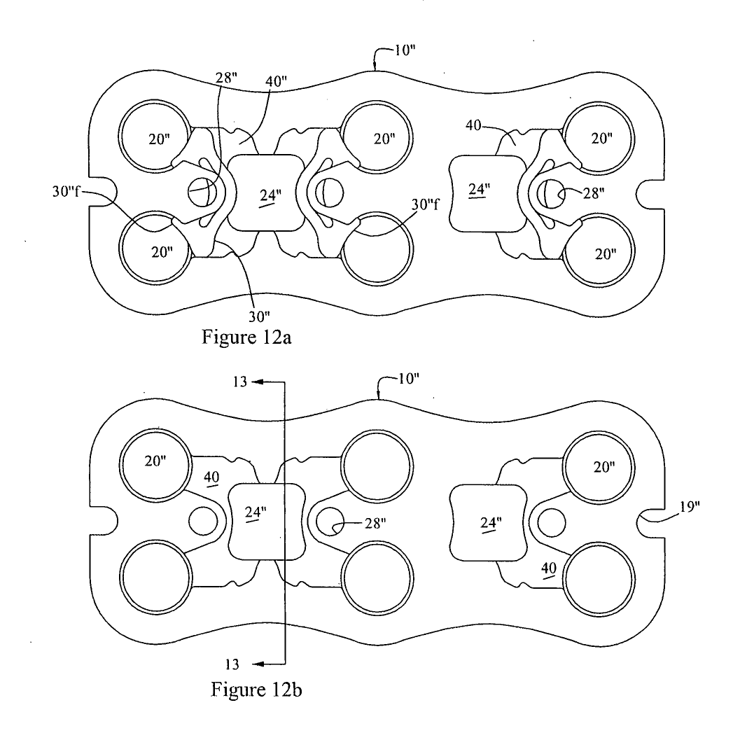

FIGS. 12-18 illustrate a modified spinal fixation plate 10″ suitable for spanning three vertebrae. In this embodiment the latch recesses and covers are machined into a solid plate dispensing with the need for welding a plate over the recesses to secure the latches in place. Like elements are marked with a double prime numeral in these figures.

The plate 10″ is formed with a depressed planar recesses 40 supporting the latch 30″. As is illustrated, each of the recesses 40 extend from a bone graft window 24″ to intersect an associated pair of screw holes 20″ along the same arc discussed with respect to the embodiment of FIG. 1 et seq. See FIG. 13.

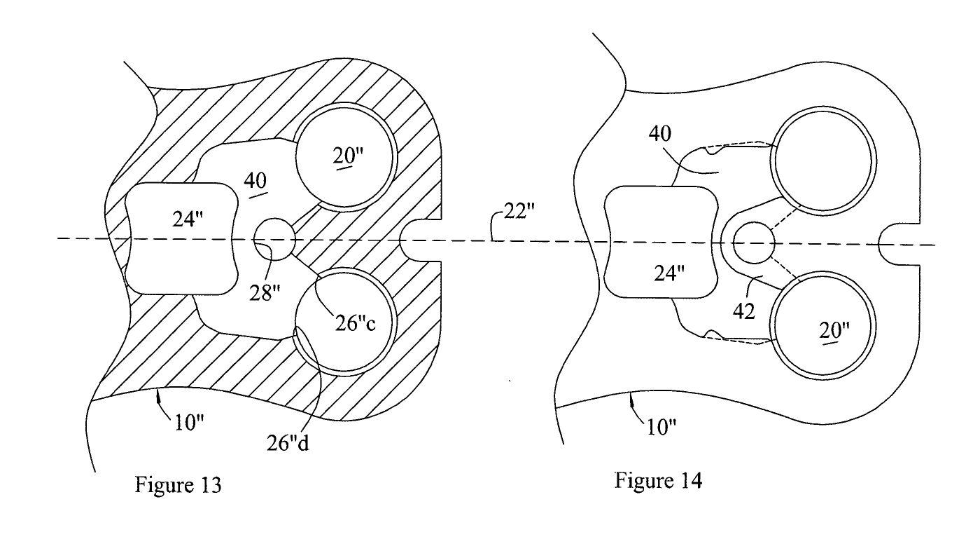

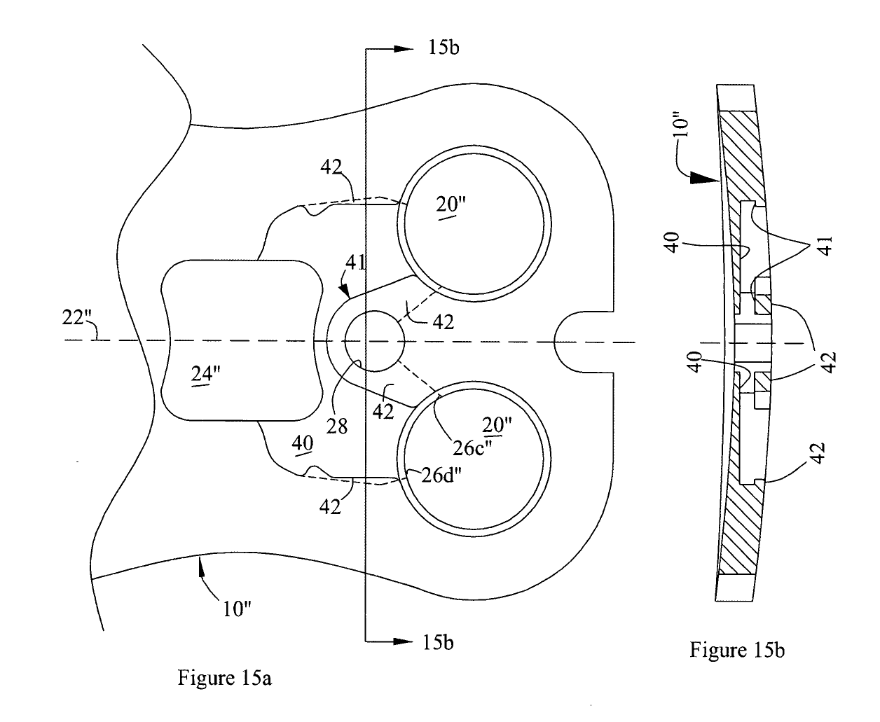

The plate 10″ is machined to form an undercut resulting in an overhang 42 represented by the area between the dashed and solid lines in FIGS. 14, 15a and 15b. This overhang, in conjunction with the recess forms a small cavity 41 extending from each screw hole along both sides of the recess, as shown.

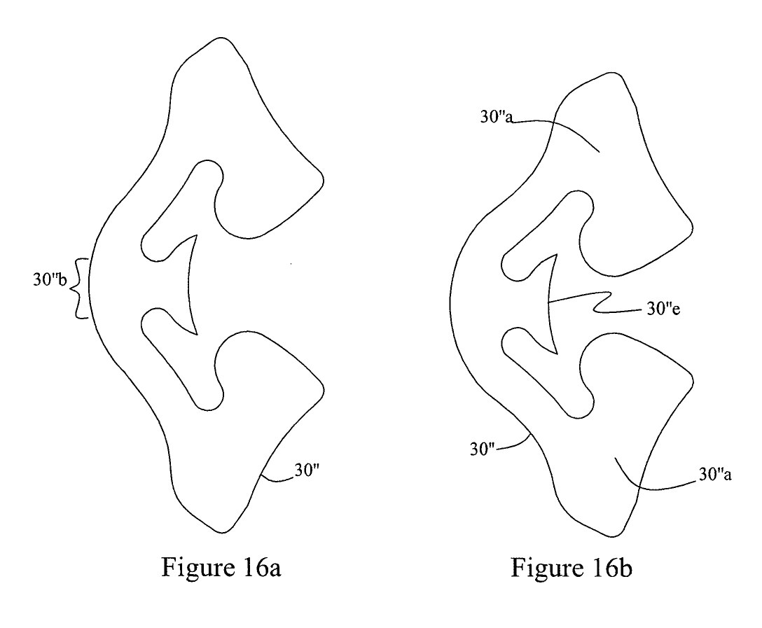

FIG. 16 illustrates the latch 30″ in its natural or uncompressed state and FIG. 16b illustrates the latch 30″ in a compressed state, necessary for installing the latch in the plate cavity.

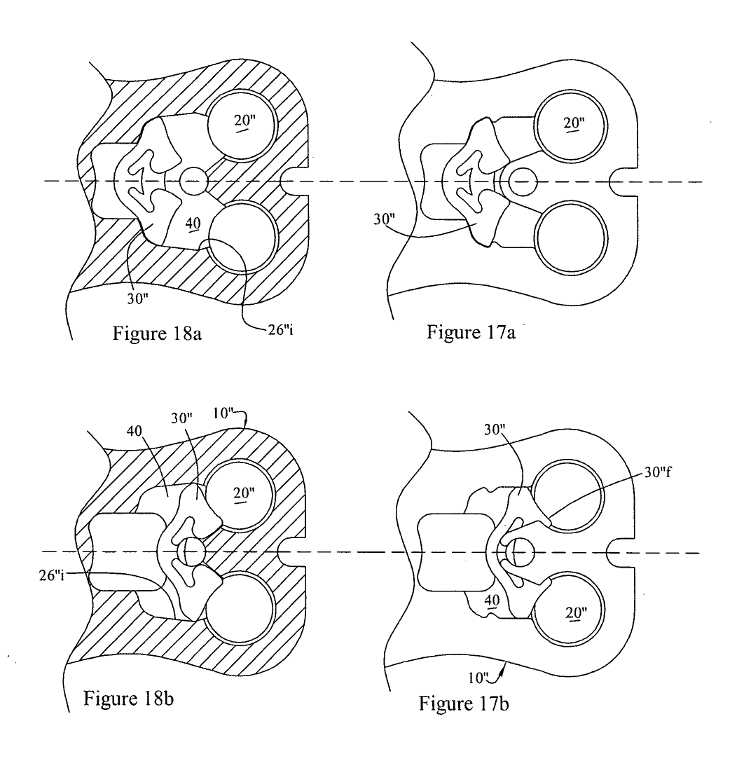

To install the latch 30″ into the cavity 41, the latch 30″ may be placed on the depressed recess 40 and then compressed and forced into the cavity so that the wings extend into the screw holes as shown in FIG. 17b. FIGS. 18a and 18b illustrate the installation step with the overhang removed via a cross-sectional view. It is to be noted that the wings 30″a of the latch are compressed beyond the maximum compression of normal use in the installation process so that the motion incurred in normal use will not force the latch back out of the plate. The procedure to retract the latch wings from the screw holes is the same as has been previously described.

There has thus been described an improved spinal/cervical fixation plate assembly which meets the challenges discussed previously. Further modifications or perhaps improvements of the plate assembly will occur to those skilled in the art without involving a departure from the spirit and scope of the invention as defined in the claims.

1. In a spinal fixation plate assembly for releasably retaining at least one bone screw having an enlarged head with a generally flat latch engaging surface and a depending threaded shaft comprising:
a fixation plate having an anterior surface, a posterior surface, a pair of spaced screw holes extending through said surfaces along generally parallel imaginary longitudinal axii each screw hole having a generally cylindrical interior wall to allow the threaded shaft to pass therethrough, but not the enlarged head and to accommodate the enlarged head of the screw therein with the latch engaging surface of the screw enlarged head extending below a lateral plane generally perpendicular to the longitudinal axis when the screw is seated, and a recess intersecting each screw hole wall above the lateral plane and along an arc beginning at a proximal point near the central plane and terminating at a distal point remote from the central plane; and
an elastically deformable generally v-shaped latch having a pair of wings joined to a central head portion through a respective arm, the latch being disposed within the recess and movable between a first position in which each wing extends into a respective screw hole above the lateral plane to contact the latch engaging surface and inhibit the egress of a screw positioned within the screw hole and a second position in which the wing is retracted from the screw hole allowing the screw to be removed, the fixation plate providing an access opening to the latch head portion to enable the latch to be manually moved to the second position.

2. The fixation plate assembly of claim 1 wherein movement of the latch central head portion in a direction away from the screw holes elastically deforms a portion of the latch to retract the wings from the screw holes allowing the screw to be removed.

3. The fixation plate assembly of claim 2 wherein the respective arms comprise a deformable portion of the latch and wherein the recess is in the form of an access opening into which the latch central head projects, whereby movement of the central head in a direction to deform the respective arms retracts the wing from the screw holes.

4. The fixation plate assembly of claim 3, wherein the recess includes a wing abutment to limit the movement of the wings into the screw holes in the first position and allows the rotation of the wings toward the central plane when the latch is transitioning to the second position.

5. The fixation plate of claim 3 wherein the recess is a cavity with two interior walls extending inwardly from the proximal point to the access opening and two exterior walls extending inwardly from the distal point.

6. The fixation plate of claim 4 wherein the wing abutment is located adjacent the distal point.

7. The fixation plate assembly of claim 1 wherein the top of the screw head comprises the latch engaging surface.

8. The fixation plate assembly of claim 1 wherein the wings are arranged to retract out of a respective screw hole in response to the insertion of the screw head into the respective screw hole.

9. The fixation plate assembly of claim 1 wherein the fixation plate is generally rectangular in shape and with said pair of screw holes and associated recesses and latch disposed on one end and a mirror image of said pair of screw holes, recess and latch located on the other end with the separate pairs of screw holes arranged to overlie the vertebral bodies to be fixed, the fixation plate defining a graft window located between each pair of screw holes.

10. A spinal fixation plate assembly for releasably retaining bone screws therein, each screw having an enlarged head with a latch engaging surface and a depending threaded shaft comprising:
a fixation plate having an anterior and a posterior surface, a pair of spaced screw holes extending through the surfaces along imaginary longitudinal axii, each screw hole having an interior surface allowing the threaded shaft to pass therethrough, but not the head and to accommodate the screw head therein; and
a v-shaped latch having a central head portion joined to a pair of wings through elastically deformable arms, the latch being carried by the plate and moveable between a deployed position in which the wings extend into the screw holes above the latch engaging surface to inhibit the egress of screws positioned within the holes and a retracted position in which the wings are retracted from the screw holes to allow the screws to be removed.

11. The fixation plate of claim 10 wherein the latch is in the deployed position in the absence of a force applied to the head and is moveable to the retracted position in response to a force being applied to the head in a direction away from the screw holes.

12. The fixation plate of claim 10 wherein the plate includes wing abutment shoulders for engaging the respective wings to limit the extent of the movement of the wings into the screw holes.

13. The fixation plate of claim 11 wherein the plate and latch are arranged so that as the latch transitions from its deployed to its retracted position the wings rotate toward each other.

14. The fixation plate of claim 13 wherein the latch is located within a cavity in the plate, the cavity having a pair of side walls associated with each wing, the side walls intersecting a respective screw hole at a proximal and distal point near and remote from the other screw hole respectively, the wing abutment shoulder being located on a side wall adjacent the distal point.

15. The fixation plate of claim 14 wherein the latch head is provided with a tool engaging surface and wherein the plate includes a head access opening for receiving said tool.

16. The fixation plate assembly of claim 7 wherein the latch is biased into the deployed position, arranged to move to the retracted position, in response to the insertion of the head of a screw into the interior of the screw hole and arranged to assume the deployed position once the latch engaging surface is located below the wing.

17. In a spinal fixation plate for releasably retaining a bone screw having an enlarged convex-shaped head in cross-section with a generally flat latch engaging surface and a depending threaded shaft, the plate comprising:
an anterior surface;
a posterior surface;
at least one generally cylindrical screw hole extending through said surface along an imaginary longitudinal axis, the screw hole having a upper concave-shaped section in cross-section for receiving the head of the screw while allowing the threaded shaft to pass therethrough and a lower neck section for accommodating the passage of the thread shaft therethrough while maintaining the screw head within the upper section;
a recess extending into the screw hole;
a formed latching member having at least one wing joined to a head portion through an elastically deformable arm, the latching member being moveable within the recess between a neutral position in which the wing thereof extends into the screw hole inhibiting the egress of a screw positioned within the hole and a stressed position in which the wing is retracted from the screw hole allowing the screw to be removed, the latching member transitioning from the neutral to the stressed position in response to movement of the head portion in a direction away from the hole.

18. The fixation plate of claim 17 wherein said at least one hole comprises two screw holes and the latching member being formed with a second wing joined to the head portion through a second elastically deformable arm with the second wing of the latching member extending into and out of the other screw hole when the latching member is in its neutral and stressed positions, respectively.

19. A spinal fixation plate having an inner and outer surface, at least two adjacent screw holes extending through the surfaces, the holes accommodating the heads of conventional bone screws therein while allowing the threaded shafts of the screws to pass therethrough, the plate characterized by:
a recess extending into each screw hole and a generally v-shaped latch positioned in the recess, the latch having a pair of wings joined to a head via a pair of elastically deformable arms, the latch being movable between a deployed position in which each of the wings extends into a respective hole to inhibit the egress of a screw positioned therein and a retracted position in which the wings are retracted from the screw holes permitting the screws to be backed out of the plate, the latch assuming the retracted position in response to movement of the head in a direction away from the holes.

20. The fixation plate of claim 19 wherein the recess is formed with a cover extending over a portion of the latch to secure the latch within the recess.

21. The fixation plate of claim 20 wherein the screw head defines a latch engaging surface and wherein the recess intersects each screw hole along a lateral plane above the latch engaging surface when a screw is seated in the screw hole.

22. The fixation plate of claim 20 wherein the latch is deformed when in the retracted position.
